(19) United States
(12) Patent Application Publication
(10) Pub. No.: US 2002/0143296 A1
(43) Pub. Date: Oct. 3, 2002

(54) Medical tube holder

(76) Inventor: Ronald D. Russo, Barrington, RI (US)

Correspondence Address: Finnegan, Henderson, Farabow,, Garrett & Dunner, L.L.P., 1300 I Street, N.W., Washington, DC, 20005-3315, US

(21) Appl. No.: 10/109,936

(22) Filed: Mar. 29, 2002

Related U.S. Application Data
(60) Provisional application No. 60/279,858, filed on Mar. 29, 2001.

(51) Int. Cl. A61M 5/32

(52) U.S. Cl. 604/180; 128/DIG. 26

(57) ABSTRACT

Systems, methods and apparatii are provided for securing a medical tube, and in particular a naso-gastric tube. According to one illustrative embodiment, a medical tube holding system is provided that includes a strip having first and second surfaces including adhesive on the first surface for attachment to a medical tube, and a tube holder including a first section for attachment to a patient, and a second section for attachment to the second surface of the adhesive strip.

### BACKGROUND OF THE INVENTION

The present application claims priority to U.S. Provisional Patent Application No. 60/279,858, filed Mar. 29, 2001.

The use of naso-gastric tubes and nasal airway tubes (hereinafter “nasal tubes”) is a common occurrence in medicine today. A naso-gastric tube is a tube fed through a patient's nostril, down the patient's esophagus, and into the patient's enteric tract (also called an “enteric tube”). A nasal airway tube is also fed through a patient's nostril, but rests in the tracheal airway, instead of extending down into the stomach. One use of a naso-gastric tube is as a means of providing enteric nutrition (such as Ensure™) to a patient. The nutrition is delivered by the tube directly into the patient's stomach.

Another use of a naso-gastric tube is as a means of gastric decompression, used to drain gas and fluid from the stomach. Levin tubes, or Salem sump tubes, fall into this category. This type of tube relieves gas and fluid pressure resulting from trauma to the patient's enteric tract from an operation or injury.

Whether using a naso-gastric tube as a feeding tube or for gastric decompression, securing and stabilizing the tube is important. If not secured properly, lateral movement or awkward positioning of the tube can irritate the patients nostril nares, especially when the tube is in place for several days or weeks.

Retraction or advancement of the tube should also be prevented. Inadvertent retraction of the feeding tube can cause the tube to retreat to the esophagus where it can dispense fluid into the lungs. Inadvertent retraction of the gastric decompression tube can cause the tube to apply suction to sensitive esophageal tissues. Furthermore, proper positioning of the naso-gastric tube is often insured by taking an x-ray of the patient; thus, once properly positioned, it is a waste of time and resources to reposition the tube if not properly stabilized.

Commonly, naso-gastric tubes are stabilized simply by taping the tube to the face of the patient. This often results in improper positioning of the tube, thereby irritating the nares. The direct application of medical tape to the patient's skin also tends to cause irritation, especially when removing the tape. Medical tape is also difficult and time consuming to apply and remove. Nasal airway tubes are also stabilized simply by taping the nose or face.

In addition to nasal tubes, there is also a need to secure and stabilize various tubes over other areas of a patient's body, e.g., intravenous tubes, gastronomy tubes, catheters, etc. The predominant means of securing and stabilizing most medical tubes is through the use of medical tape, whose disadvantages have been mentioned above.

### SUMMARY OF THE INVENTION

Systems, methods and apparatii are provided for securing a medical tube, and in particular a naso-gastric tube. According to one illustrative embodiment, a medical tube holding system is provided that includes a strip having first and second surfaces including adhesive on the first surface for attachment to a medical tube, and a tube holder including a first section for attachment to a patient, and a second section for attachment to the second surface of the adhesive strip.

According to another illustrative embodiment, a method of securing a medical tube is provided that includes securing a first surface of a strip to the medical tube such that a second surface of the strip is exposed; attaching one end of a tube holder to a patient; and attaching a second end of the tube holder to the second surface of the strip to fixedly attach the tube to the patient.

It is to be understood that both the foregoing summary and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a frontal plan view of one illustrative embodiment of a medical tube holder consistent with the principles of the present invention;

FIG. 2 is a perspective view of a strip used in conjunction with the medical tube holder of FIG. 1 to secure a medical tube;

FIG. 3 is a rear plan view of the tube holder of FIG. 1;

FIG. 4 is a side view of the tube holder of FIG. 1;

FIGS. 5 and 6 show the adhesive strip of FIG. 2 applied to naso-gastric tubes mounted in a patient's left and right nostril, respectively;

FIGS. 7 and 8 show the tube holder of FIG. 1 being applied to secure the naso-gastric shown in FIGS. 5 and 6, respectively; and

FIG. 9 shows a bottom cross-sectional view of the tube holder of FIG. 1 and the strip of FIG. 2 used in a medical tube holding system to secure a naso-gastric tube.

### DETAILED DESCRIPTION

Reference will now be made in detail to several illustrative embodiments of the present invention, examples of which are shown in the accompanying drawings. Wherever possible, the same reference numbers will be used throughout the drawings to refer to the same or like parts.

The present invention enables medical tubes, and in particular naso-gastric tubes, to be securely held in a central location within the nostril to prevent irritation and necrosis. The tube holder described herein also readily adapts to tubes intubated in either the right or left nostril, and permits easy removal and replacement of the tube holder. Although the illustrative embodiment disclosed herein is directed towards securing naso-gastric tubes, the principles of the invention can also be applied to secure different types of medical tubes attached at various locations on a patient's body.

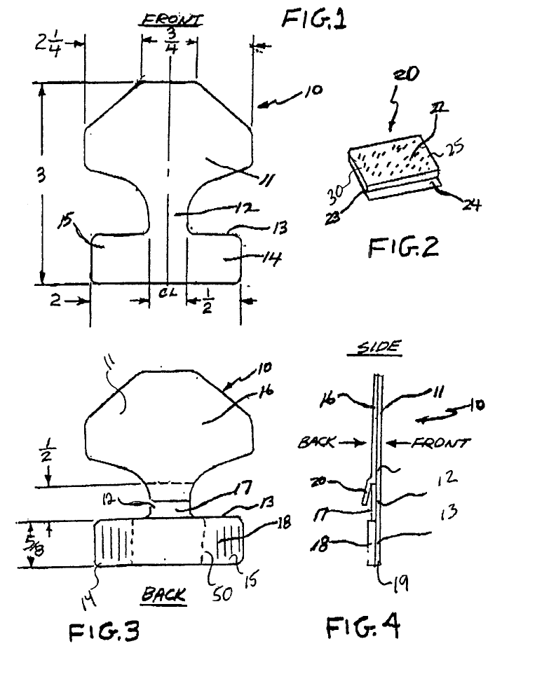

FIGS. 1-9 show an illustrative embodiment of a medical tube holder system, apparatii and method designed to secure a naso-gastric tube consistent with the principles of the present invention. The medical tube holder system includes a holder 10 (FIG. 1) and an adhesive strip 20 (FIG. 2). The tube holder 10 includes a first section 11 for adhering to a patient's nose, a narrow neck section 12, and a tab section 13. The tab section 13 includes a right tab 14 and a left tab 15.

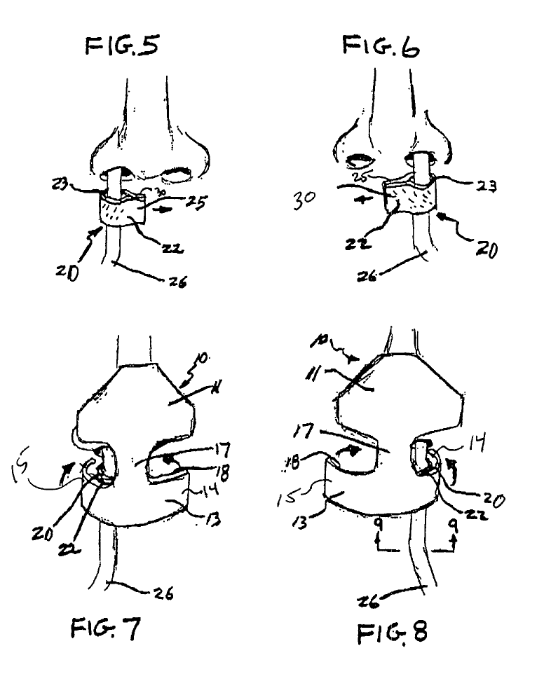

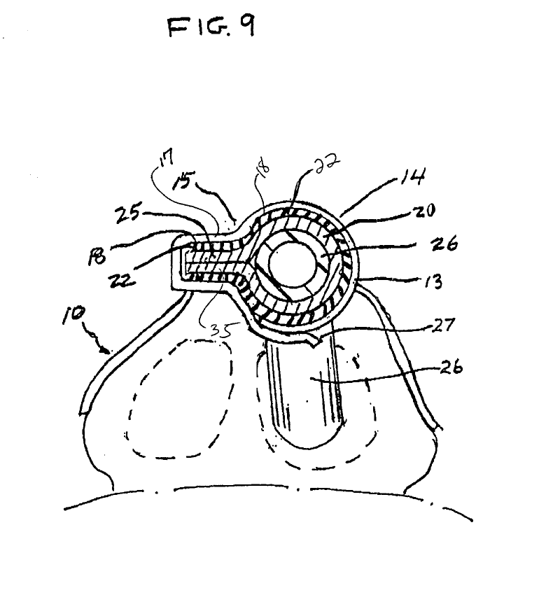

The tube holder is formed by a first base layer 19 (FIG. 4) which can be, by way of non-limiting example, a polyurethane film, 3M® Durapore®, or Transpore®. 6 mils to 10 mils has been found to be an appropriate thickness for base layer 12. Attached to the base layer is a release liner 16 which covers an adhesive mounted on section 11 which facilitates attachment of section 11 of the tube holder 10 to a patient's nose.

Also attached to the base layer 19 is a reinforced polyester mylar laminate 17 used to strengthen the relatively narrow neck section 12. 3 to 10 mils has been found to be an appropriate thickness for laminate 17. The release liner 16 preferably includes a section 20 that extends partially over laminate 17 to facilitate the removal of the release liner to expose the adhesive disposed on the base layer 19. Also attached to the base layer 19 is a Velcro® loop material 18 laminated onto tab section 13. Although the invention is not limited to any particular dimensions or sizes, preferred dimensions are shown in inches in FIGS. 1 and 3.

The adhesive strip 20 of the medical tube holder system includes a Velcro® hook surface 22 laminated to a base having a semi-permanent adhesive 23 disposed on the side opposing the hook surface 22. A release liner 24 which extends past an edge of the strip (FIG. 2) covers the adhesive 23 prior to use.

As shown in FIGS. 5-9, to secure a naso-gastric tube 26 to a patient, first the adhesive strip 20 is wrapped around tube 26 such that adhesive layer 23 securely adheres to the tube 26 and ends 25 and 30 of the strip extend past the tube and mate with one another in adhesive-to-adhesive contact of surface 23, with ends 25 and 30 directed to the non-intubated nostril (FIGS. 5 and 6). Accordingly, the hook surface 22 is exposed about the tube 26.

To secure the tube, the tube holder 10 is then attached about the tube and to the nose of the patient. Release liner 16 is removed to expose the non-permanent adhesive disposed on base layer 19 to permit section 11 to be attached to the patient's nose. Additionally, either left tab 15 (FIG. 7) or right tab 14 (FIG. 8) wraps around tube 26 and strip 20 attached thereabout such that loop material 18 contacts and adheres to hook material 22. As best shown in FIG. 9, the tab 15 distal from the tube 26 is also wrapped around the exposed hook surface 22 of the adhesive strip to adhere to the side 35 of the adhesive strip facing the patient. Scoring 50 can also be provided in tabs 14 and 15 to facilitate folding the tabs into contact with strip 10, and help prevent the tabs from biasing away from contact with strip 10, as described in co-pending Ser. No. 09/567,780, filed May 9, 2000. Thus, the tab 13 and strip 20 form a soft, flexible and secure mechanical connection which does not stress or crush the tube as would, for example, a holder that uses a clamping action.

The reinforced laminate 17 prevents the tab portion 13 from moving or torqueing such that once engaged lower tab portion 13 forms a secure stabilized engagement with strip 20. The above-described tube holder system clearly holds the tube in a central location within the nostril to prevent irritation and necrosis. The holder 10 is also a one size fits all device and that it will securely retain all sizes of tubes up to 18 French in outer diameter. The tube holder also readily adapts to tubes intubated in either the right or left nostril.

The tube holder also permits easy removal and replacement. The tabs 14 and 15 can easily be disengaged by pulling up on end 27 (FIG. 9) of tab 15 and disengaging the loop-to-hook engagement. Holder 10 can be moved and replaced with a new holder while still using the existing strip on the tube, enabling the area above the nose to be cleaned easily. The hook and loop engagement of tab 13 to strip 20 is advantageous over prior art adhesive holders in that the hook and loop engagement does not depend upon an adhesive attachment which can release as the adhesive joint degrades due to nasal or oral secretions, or from oxygen delivered to the patient by mask or oxygen cannula. The layered construction of the holder also makes automatic inline fabrication inexpensive.

It will be apparent to those skilled in the art that additional various modifications and variations can be made in the present invention without departing from the scope or spirit of the invention. For example, although the hook and loop attachment surfaces can be reversed on the strip 20 and holder 10, and/or other appropriate fastening means can be used.

Other embodiments of the invention will be apparent to those skilled in the art from consideration of the specification and practice of the invention disclosed therein. It is intended that the specification and examples be considered as exemplary only, with a true scope and spirit of the invention being indicated by the following claims.

What is claimed is:

1. A medical tube holding system comprising:
a strip having first and second surfaces, the strip including adhesive on the first surface for attachment to a medical tube; and
a tube holder including a first section for attachment to a patient, and a second section for attachment to the second surface of the adhesive strip.

2. The tube holding system of claim 1, further comprising fastening means to facilitate attachment between the second section and the second surface of the adhesive strip.

3. The tube holding system of claim 2, wherein the fastening means includes hook and loop materials on the second surface of the adhesive strip and the second section, respectively.

4. The tube holding system of claim 1, wherein the first section includes a non-permanent adhesive for attachment to a patient's skin.

5. The tube holding system of claim 1, wherein the first section is attached to the second section via a narrow section relative to the first and second sections.

6. The tube holding system of claim 5, further comprising a material attached to the narrow section to provide support.

7. The tube holding system of claim 1, wherein the second section includes a tab adapted to be wrapped around the adhesive strip to secure the medical tube.

8. The tube holding system of claim 7, wherein the tab includes right and left tab ends, and one of the right and left tab ends is adapted to wrap around the front of the tube distal from the patient and the other of the right and left tab ends is adapted to wrap around the back of the tube proximal to the patient, depending on whether the tube extends from a right or left nostril of the patient.

9. The tube holding system of claim 1, wherein the first section is constructed and arranged to attach to the patient's nose.

10. The tube holding system of claim 9, further comprising fastening means to facilitate attachment between the second section and the second surface of the adhesive strip.

11. The tube holding system of claim 1, wherein the second section of the tube holder is adapted to be releasably attached to the strip to permit removal and replacement of the tube holder.

12. The tube holding system of claim 11, wherein the adhesive is permanent adhesive.

13. A method of securing a medical tube, comprising the steps of:
(A) securing a first surface of a strip to the medical tube such that a second surface of the strip is exposed;
(B) attaching one end of a tube holder to a patient; and
(C) attaching a second end of the tube holder to the second surface of the strip to fixedly attach the tube to the patient.

14. The method of claim 13, wherein step (B) includes attaching the one end to the nose of the patient.

15. The method of claim 13, further comprising the steps of:
(D) removing the tube holder from the patient and the strip;
(E) disposing of the tube holder; and
(F) repeating steps (B) and (C) with a second like tube holder.
